(19) United States
(12) Patent Application Publication
(10) Pub. No.: US 2017/0268037 A1
(43) Pub. Date: Sep. 21, 2017

(54) METHODS AND SYSTEMS FOR CELL SEPARATION USING MAGNETIC-AND SIZE-BASED SEPARATION

(71) Applicant: FLUXION BIOSCIENCES, INC., SAN FRANCISCO, CA (US)

(72) Inventors: CRISTIAN IONESCU-ZANETTI, BERKELEY, CA (US); MICHAEL SCHWARTZ, OAKLAND, CA (US); CHRISTINE TING FU, SAN FRANCISCO, CA (US); RONG ANDREA FAN, REDWOOD CITY, CA (US)

(73) Assignee: FLUXION BIOSCIENCES, INC., SAN FRANCISCO, CA (US)

(21) Appl. No.: 15/310,083

(22) PCT Filed: May 15, 2015
(86) PCT No.: PCT/US15/31217

§ 371 Date: Nov. 9, 2016

Related U.S. Application Data
(60) Provisional application No. 61/993,431, filed on May 15, 2014.

(51) Int. Cl. C12Q 1/68; B03C 1/28; B03C 1/01; B03C 1/033; B01L 3/00; B03C 1/30

(52) U.S. Cl. CPC C12Q 1/6806; B01L 3/502761; B03C 1/30; B03C 1/01; B03C 1/0332; B03C 1/288; B01L 2200/0652; B01L 2400/043; B03C 2201/18; B03C 2201/26

(57) ABSTRACT

A method is provided including coupling magnetic beads to a population of cells in a fluid sample to form magnetically-labeled cells, magnetically separating the magnetically-labeled cells from non-magnetically-labeled cells in the fluid sample, and separating target cells from non-target cells of the magnetically-labeled cells based on a size difference between the magnetically-labeled target cells and the magnetically-labeled non-target cells. A microfluidic device is provided including a fluidic pathway traversing a magnetic isolation region and a size-based isolation region. The magnetic isolation region includes a magnet positioned to separate magnetically-labeled cells from non-magnetically labeled cells in the magnetic isolation region. The size-based isolation region includes a separator configured to separate cells less than a threshold size from cells greater than a threshold size.

### CROSS-REFERENCE TO RELATED APPLICATIONS

This application claims the benefit of the earlier filing date of U.S. Provisional Application No. 61/993,43 filed May 15, 2014, and entitled “MULTIPARAMETRIC METHODS AND SYSTEMS FOR CELL SEPARATION INCLUDING DIAGNOSTIC APPLICATIONS”, which application is hereby incorporated by reference in its entirety for any purpose.

### BACKGROUND

The separation of target cells from a biological fluid (such as blood, urine, saliva) is an important area of development, with applications in both the clinical diagnostic and the basic research fields. For a number of applications, separation is performed by applying differential forces to the positive fraction (cells of interest) as compared to the negative fraction (background cells). Devices have been described where various physical properties, is size, motility, electric charge, electric dipole moment, optical qualities, and magnetic susceptibility have been used to separate specific cells or molecules from these mixtures. Another approach has been to separate cells based on binding of specific surface markers. For example surfaces of microfluidic channels have been patterned with a variety of antigen molecules; a subset of the cell population then interacts with the surface and gets immobilized by binding the surface antigen. Another approach taken has to selectively bind beads of a paramagnetic material to the cells of interest, typically via a surface marker present at the cell membrane. The positive fraction is then separated by bringing the labeled cells into a region of increased magnetic field gradient by either placing a magnet close to the cell suspension or microfluidic channel, or by using an external magnet in order to magnetize structures that have be incorporated in the microscale device and amplify the field gradient in an adjacent region of space. Various macroscale and microscale devices have been presented that are aimed at separation of magnetically labeled species.

### SUMMARY

An example method includes coupling beads to a population of target cells based on antibody binding in a fluid sample to foam target cell-bead aggregates having a larger size than a population of non-target cells in the fluid sample. The method also includes separating the target cell-bead aggregates from the non-target cells based on a size difference between the target cell-bead aggregates and the non-target cells.

Another example method includes coupling magnetic beads to a population of cells in a fluid sample to form magnetically-labeled cells, wherein certain of the magnetically-labeled cells are target cells and others of the magnetically-labeled cells are non-target cells. The method further includes magnetically separating the magnetically-labeled cells from non-magnetically-labeled cells in the fluid sample. The method also includes separating the target cells from the non-target cells of the magnetically-labeled cells based on a size difference between the magnetically-labeled target cell-bead aggregates and the magnetically-labeled non-target cells.

An example microfluidic device includes an input, an output, and a fluidic pathway extending between the input and the output. The fluidic pathway traverses a magnetic isolation region and a size-based isolation region. The magnetic isolation region includes a magnet positioned to separate magnetically-labeled cells from non-magnetically labeled cells in the magnetic isolation region. The size-based isolation region is downstream of the magnetic isolation region and includes a separator configured to separate cells less than a threshold size from cells greater than a threshold size. The threshold size is greater than a size of some magnetically-labeled non-target cells but less than a size of some magnetically-labeled target cells. In some examples, the threshold size is greater than a size of a majority of magnetically-labeled non-target cells but less than a size of a majority of magnetically-labeled target cells.

This summary is provided to aid understanding, and one of skill in the art will understand that each of the various aspects and features of the disclosure may advantageously be used separately in some instances, or in combination with other aspects and features of the disclosure in other instances. Accordingly, while the disclosure is presented in terms of examples, it should be appreciated that individual aspects of any example can be claimed separately or in combination with aspects and features of that example or any other example.

### INCORPORATION BY REFERENCE

All publications and patent applications mentioned in this specification are herein incorporated by reference in their entirety and for any purpose to the same extent as if each individual publication or patent application was specifically and individually indicated to be incorporated by reference.

### BRIEF DESCRIPTION OF THE FIGURES

A better understanding of the features and advantages of example methods, systems, and compositions may be obtained by reference to the following detailed description that sets forth illustrative embodiments, in which the principles of methods, compositions, devices and apparatuses are utilized, and the accompanying drawings of which:

FIG. 1 presents an example sample workflow for separating cells out of a fluid sample, such as a blood sample, in accordance with the present disclosure. In some examples, after separation of the buffy coat from the blood sample, beads are coupled to the cells of interest either in or outside the device. Size based separation may be performed based on the effective size of the coupled beads and cells using a separator, such as a microfluidic device, a filter substrate, or other device. Cell content recovery of the substrate using fluid flushing or cell lysis may be followed by molecular profiling of said cells.

FIG. 2 presents a schematic of an example separation principle in accordance with the present disclosure. In this example, target cells may be the same size as non-target cells. Beads may be bound to target cells to increase their apparent size relative to the non-target cells (FIG. 2A). The non-target cells may flow through a size based separation device, referred to herein as a separator, such as a filter, whereas the target cells and beads bound thereto may be captured by the separator, resulting in the target cells being separated from the non-target cells (FIG. 2B).

FIG. 3 presents an example sample Workflow for combining magnetic separation with bead-enhanced size separation in accordance with the present disclosure. After separation of the huffy coat from a blood sample, large magnetic beads are coupled to the cells of interest either in or outside the device. A first separation is performed using magnetic forces based on magnetic bead binding to the target cell population. The resulting enriched population is subjected to a second size based separation using a separator, such as but not limited to a microfluidic device, a filter substrate, or other device, resulting in a high purity level of target cells, such as tumor cells. The cells may then be analyzed via molecular profiling.

FIG. 4 presents a schematic distribution of example cell characteristics and separation based on a combination of immuno-magnetic separation and size-based separation for circulating tumor cells (CTCs) in accordance with the present disclosure. To start, CTCs have an average size larger than white blood cells (WBCs), but there is still significant overlap, preventing efficient size-based separation (FIG. 4A). A bead binding step, whereby cells preferentially bind CTCs, leads to the reduction in the size overlap between the two populations, and enables efficient size-based separation (FIG. 4B). Optionally, much higher purity may be achieved by utilizing the same beads preferentially bound to CTCs to immunomagnetically deplete the WBC population before the size-based separation. This results in a WBC population in some examples thin is much smaller but across the same size spectrum going into the last size-based separation (FIG. 4C).

FIG. 5 presents a schematic overview of an example microfluidic device designed to perform particle separation in accordance with the present disclosure. The device generally includes two inputs and two outputs, and a fluidic pathway extending between the inputs and the outputs. The device also may include two regions: a first separation region (which may be referred to as a magnetic cell isolation region) and a second separation region (which may be referred to as a size-based cell isolation region). In the first separation region, the positive and negative fractions are magnetically separated due to, for example, magnetic bead binding to the target (and some non-target) cells. In the second separation region, the cells are separated based on size.

FIG. 6 presents a schematic of a separation workflow that includes a removable section that forms at least part of the wall of a fluidic pathway (e.g. microfluidic channel) and a separator, such as a filter substrate, for size-based separation in accordance with the present disclosure. In this vertical cross section, the removable section is positioned between the magnet and the separation chamber (FIG. 6A). Due to magnetic forces acting on labeled cells, the cells are immobilized on the bottom surface of the removable substrate and removed from the device along with said substrate (FIG. 6B). Post separation, cells are recovered by removing the substrate (FIG. 6C) and placed, for example, via pipetting on top of a filter substrate (FIG. 6D) for further removal of the non-target population. In this example, the size-based separator is a filter substrate, and separation is based on size exclusion.

FIG. 7 presents an example workflow whereby cancer patient blood samples are obtained, and enriched for circulating tumor cells by using both immunomagnetic and physical properties (e.g. size) in accordance with the present disclosure. The cells are then lysed and nucleic acids are then extracted from said sample, followed h processing via NGS. The resulting DNA abnormality information may be assembled into a report interpreting said information, the report being used as a diagnostic or to aid in monitoring and treatment decisions for cancer patients.

FIG. 8 presents an example workflow whereby cancer patient blood samples are obtained, and enriched for circulating tumor cells by using both immunomagnetic and physical properties (e.g. size) in accordance e with the present disclosure. The cells are then lysed and processed for RNA extraction. The presence of a number of different expression markers is determined via qPCR, expression arrays, digital PCR and/or RNA-seq. Finally, the information ma be used to generate a patient-specific expression profile or score, to be used as a diagnostic and/or prognostic to aid in monitoring and treatment decisions for cancer patients.

FIG. 9 presents an example workflow whereby cancer patient blood samples are obtained, and enriched for circulating tumor cells by using both immunomagnetic and physical properties (e.g. size) in accordance with the present disclosure. The cells are then lysed processed for RNA extraction. The presence of circulating tumor cells is determined using a number of different expression markers via qPCR, expression arrays, and/or PCR. If the sample is determined to contain CTCs (CTC positive) the sample may then be analyzed via NGS to further characterize any DNA abnormalities.

### DETAILED DESCRIPTION

Examples described herein include methods of separating target cells, such as rare circulating tumor cells, from a liquid sample, such as a liquid biopsy, and using said cells to determine a molecular profile for a patient, such as a cancer patient. Example systems and methods are also described for the separation of biological material selectively bound to beads using size-based separation in combination with other methods, in particular immunomagnetic separation. A number of advantageous device designs and methods are described whereby the target species is separated from the non-target species. Some example methods utilize combinations of one or more of the following forces and cell properties: the size of the bead and cell complex, target cell size, or target cell mechanical properties, magnetic forces, and the size of the bound beads. The designs are adapted for the labeling, separation, enumeration, and recovery of target cells from a negative background with high purity and high recovery rate, including purities that are high enough to enable effective analysis via next generation sequencing. Examples may also include a mechanism by which molecular information resulting from the analysis of enriched tumor cells in a liquid biopsy is used as a diagnostic in improving treatment decisions for cancer patients.

Examples described herein relate to improved systems for the separation of cells from fluid samples, such as biological fluids. Some embodiments include bead dependent size based separation, whereby the apparent size of cells is increased via the specific binding of beads to target cells, but not other non-target (e.g. background) cells. The cell size may be further amplified by binding a second bead type to the first bead type (which is already bound to target cells). The quality of separation in terms of rapture efficiency, percent purity, and percent recovery may be increased by rising a combination of modalities to enable separation of the biological material. Some examples include a combination of magnetic separation and size based separation, whereby apparent cell size is increased by the presence of beads that are specifically bound to target cells. Some examples include a removable substrate which may facilitate recovery of cells whereby the cells can be extracted from the device, such as from a separation chamber. Example systems presented also have the ability to characterize cells via molecular analysis methods including qPCR, sequencing, digital PCR, and/or expression profiling. The high cell purity which may be obtained by combining size based separation with immunomagnetic separation makes possible in sonic examples the routine analysis of tumor cells from liquid biopsies via next generation sequencing (NGS). The proposed diagnostic methods may also be used in lieu of tissue biopsy-based molecular diagnostics in some examples, such as where obtaining a biopsy is difficult or impossible, or to aid therapy selection in patients that are about to start a new course of therapy.

A number of devices and methods have been previously presented for the separation of cells or other biological materials of interest from a heterogeneous mixture. Further, a number of potential uses have been presented, especially related to the analysis of rare circulating tumor cells (CTCs) from cancer patients in order to predict prognosis and assess treatment efficacy for cancer patients. Both macroscopic and micro-scale devices have been envisioned, and a number of particle properties used to enable separation of a positive fraction of cells from a larger population. A variety of cell properties have been used to separate populations, including: fluorescence, cell binding to a substrate, magnetic properties, cell binding to magnetic beads/magnetic forces, inertial properties coupled with acoustic waves, optical and electrical properties of the cells. Previously presented systems still may fall short for a number of applications, especially where the positive fraction represents a very small percentage (<0.1%) of the total population. The previously presented approaches referenced have a number of drawbacks which may be addressed, in whole or in part, by examples described herein. Drawbacks of conventional systems and advantages of systems described herein are presented by way of example and to facilitate understanding of aspects of examples described herein. The description of drawbacks and advantages is not intended to be limiting—it is to be understood that not every example described herein may address all, or even any, of the drawbacks of conventional systems, and not every example described herein may have all, or even any, of the described advantages. Disadvantages noted in conventional systems are as follows:

1. Low capture efficiency. For example, a number of cells are lost during either the transfer steps, along a flow path, or eluted downstream along with the negative fraction of the sample. Some examples from the conventional systems include: Macroscopic systems where different parts of the cell sample experience very different forces because of the geometry. Microscale flow systems where cells are bound to the channel wall, which required that the cells of interest come into intimate contact with channel wall; this requirement leads to a number of cells not binding to the functionalized walls and being eluted downstream along with the negative fraction.

2. Low purity. For a number of downstream analysis modalities, in particular next generation sequencing (NGS) it is important to present a highly concentrated sample of the positive fraction cells, without contaminating negative fraction cells. This is an especially challenging parameter to optimize for samples where the positive fraction represents a very small percentage of the overall sample, such as below 1/1000 or below 1/10E6 cells. Macroscopic systems, for example, cannot apply a constant separation force in the separation region, resulting in the inclusion of negative cells in the separated sample. One example are systems where both sedimentation and magnetic forces results in pulling sample cells to the bottom of a receptacle, in which case there is a non-zero probability of negative fraction cells being pulled out of a flow stream. For immunomagnetic separation, a small fraction of the magnetic beads will bind non-specifically to background cells that are not part of the target population, leading to the presence of a significant contaminating fraction, especially when the negative fraction population is much higher with respect to the positive fraction. Low purity samples preclude a number of desirable molecular analysis modalities, including next generation sequencing.

3. Inability to recover the positive fraction for molecular analysis. Systems that separate cells by binding the positive fraction to the flow channel walls, for example, cannot remove the bound cells easily for downstream analysis. Either cell lysis or a harsh elution step are required if cell recovery is desired. This decreases purity, reduces availability of viable positive fraction cells, decreases the final density of cells in the recovered samples, and increases the complexity of device operation.

4. Inability to recover viable cells. For example, in systems such as sorting flow cytometers, high flow velocities are required, whereby shear forces significantly impact the viability of separated cells. Other systems require the use of either fixed cells or a lysis huller to elute the, cell contents. In both cases, the recovered cells are no longer viable, complicating protein analysis and eliminating the option of performing mRNA based analyses that require viable cells and negating the ability to subsequently culture separated cells off-chip. In addition, for certain classes of cells such as CTCs the positive fraction cells are the only ones that divide in a short time scale; therefore, cell culture will naturally lead to a much higher purity sample and enable proteomic work.

5. Inability of the user to customize the capture methodology. The data from separations and analysis of the positive fraction cells may be useful in modifying or improving the capture criteria; therefore it is important for the user to have the ability of customizing the surface marker (or set of markers) used to capture positive fraction cells and other aspects of the capture methodology. For a number of previously presented systems, the capture methodology is fixed by the device manufacturer (for example capture onto solid substrates). Further, for systems where intrinsic physical properties of the cells determine the capture force (e.g. Sedimentation based capture, optical traps, acoustic focusing, electrophoretic phenomena, etc.) such forces cannot be tuned by the user to fit the need application—for example, for size-based separation (filtering) cell sizes have a natural distribution, including an overlap between different cell subpopulations. The proposed immunomagnetic separation, or combination of immunomagnetic separation with other separation modalities addresses this issue by allowing custom panels of markers to be used for bead-based separation.

6. Long run times. For various reasons (cell viability, cell settling, workflow considerations, etc.) it is important that the separation reaction be completed w/in a reasonable period of time, preferably under 1 hour, and more preferable under 15 min. For a lot of the existing systems, the full separation protocol lasts significantly longer. For example, some proposed microfluidic systems necessitate passing a full blood sample (7.5 ml) through a microscale channel of small dimensions (<1 mm). Therefore, processing the entire blood sample often takes >1 h. Examples described herein may reduce the required time dramatically.

7. Processes not amenable to automation. A number of systems currently under development are not compatible with standard fluid handling equipment, and, in other cases, don't have the ability to run multiple samples in parallel. Finally, the throughput and sample to sample contamination are important issues limiting overall throughput of separation reactions. Examples described herein provide a system where the fluid path is fully disposable (e.g. little or no cross-contamination sample to sample), can run multiple samples in parallel, and uses a. standard consumable format compatible w/existing fluid handling. equipment. Approaches that share pipetting steps, for example, always have a potential for cross contamination.

8. Large fluid volumes/large dilution for the separated fraction. Another important consideration is the volume of fluid per cell in the positive sample post separation. For certain analysis modalities, such as genotyping via PCR, it is important that the, cells are separated into a low fluid volume. However, for flow-through separation methods like FACS (fluorescence activated cell sorting), it is very difficult to separate the cells of interest into volumes below a few μL per cell. For low cell numbers in a large background, the total volume of the positive fraction is often in the mL range. The example devices and methods described herein make possible the separation of cells into a few μL for all positive fraction cells, or down to 0.01 μL per well for 100 separated cells.

Examples described herein may address one or more of the above disadvantages through novel device designs, methods, and systems.

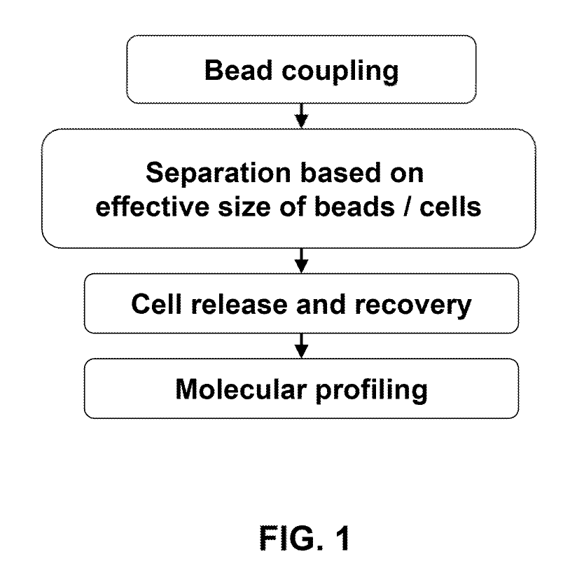

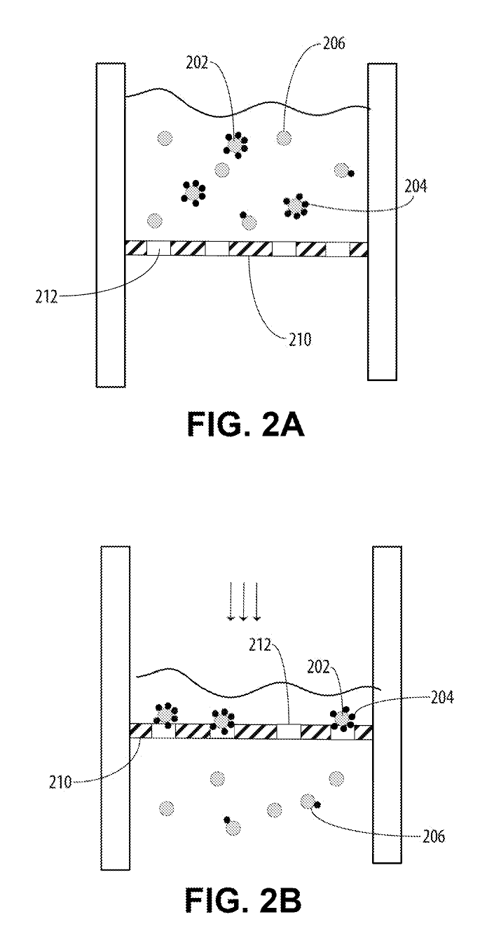

Example methods are provided which may be used for the separation of a sub-population of cells from a larger mixture based on bead-activated size exclusion. A set of functionalized beads are mixed with the whole population, causing selective binding to the target population, providing said target population expresses a known surface marker. If further size amplification is desired, a second set of beads that bind the first bead type may be added to the mixture, binding to beads that are already decorating target cells. After the bead coupling step, size-based separation using a separator device such as, but not limited to, a filter or a microfluidic element is performed. The target sub-population is retained based on the combined size of bound beads and cells, in a process termed bead-activated size exclusion. A sample workflow using this method is presented in FIG. 1, with a schematic representation presented in FIG. 2. A compelling application of this proposed isolation modality includes the lysis of the target cell population, extraction of the nucleic acids and molecular analysis of the cell population. One feature of the of some example methods is the combined use of both magnetic bead affinity-based separation and size separation on the same cell population. Another feature is the use of the same magnetic beads for both magnetic separation and the selective increase in apparent size of the bead bound cells; this size increase is then used to enhance size-based separation.

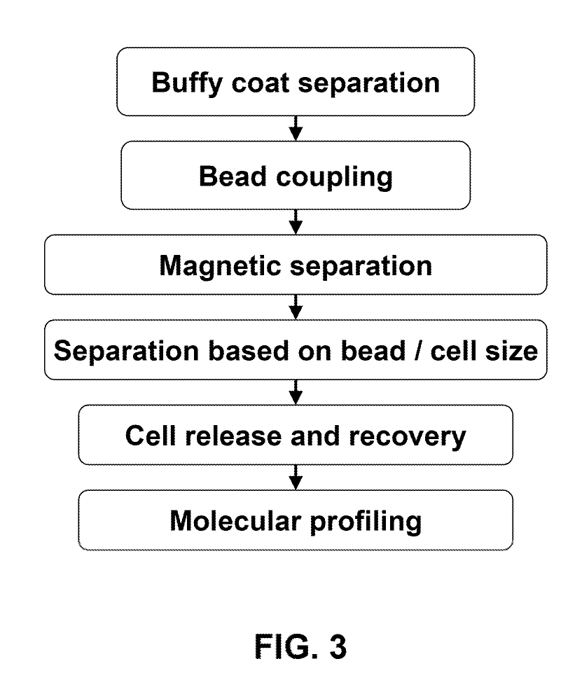

A particularly compelling workflow may combine orthogonal separation modalities like magnetic separation and size-based separation. A first separation is performed using magnetic forces based on magnetic bead binding to the target cell population. The resulting enriched population is subjected to a second size based separation using a separator, such as a microfluidic device, a filter substrate, or other separation mechanism, resulting in tumor cells at high purity. Cells may then be analyzed via molecular profiling. (See, e.g. FIG. 3).

Example systems are provided for the separation of a cell sub-population (e.g. a rare cell population) from a larger mixed sample of cells in suspension, the system may include: functionalized beads that bind an antigen specifically expressed by the target population (thereby increasing the apparent cell size), and a size-based separation device. Said system may also contain: a separation chamber, a magnetic field source, and functionalized magnetic beads that bind an antigen specifically expressed by the target population (thereby increasing the apparent cell size). The general mode of operation of such a proposed system may include one or more of the following steps:

1. The functionalized beads are selectively bound to a sub-population of cells expressing the antigen of interest (or a collection of antigens of interest). This step can either be done on or off-chip, and precautions should be taken to minimize non-specific binding of beads to negative fraction cells. When the beads bind to the target cells, the distribution of apparent cell sizes changes, such that target cells form a population with a higher apparent size as compared to the background (non-target) population.

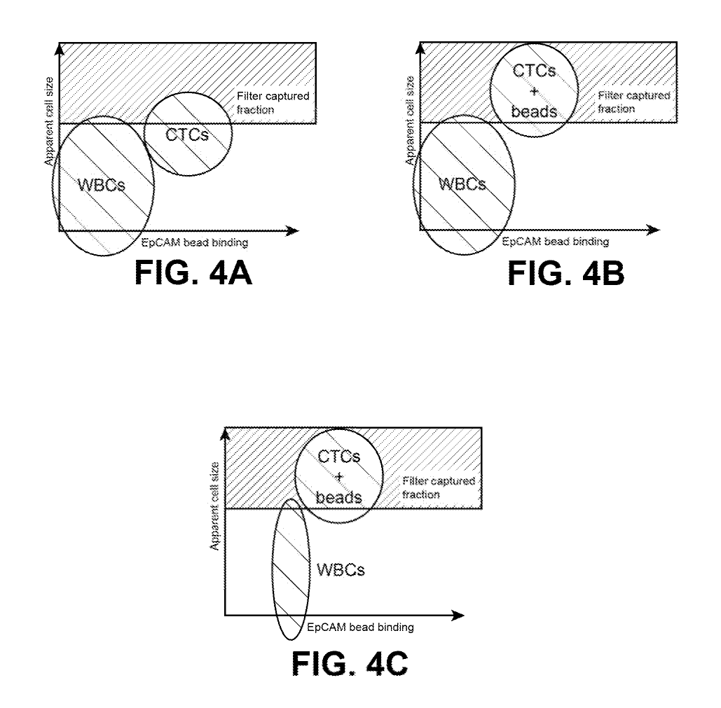

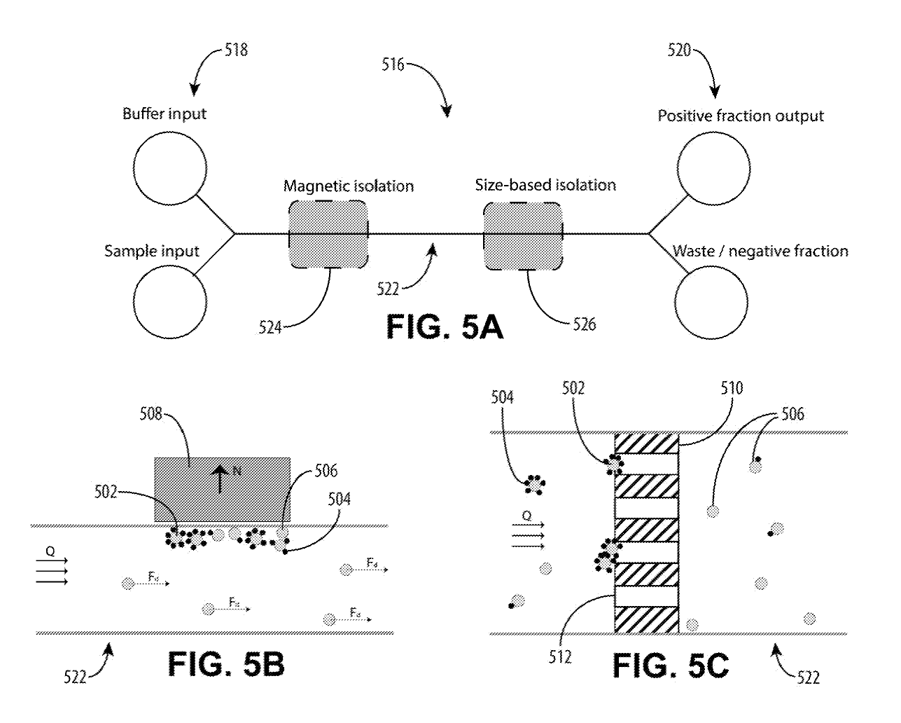

2. Cells are separated based on overall apparent size, which is a combination of inherent cell size and the size of bound beads. Size-based separation can be performed using either a filter membrane type setup, or a microfluidic channel designed to separate cells based on size. Example microfluidic channels may contain obstacles (see, e.g., FIGS. 4 and 5), or a curved/spiral shape that, at a suitable flow rate, separates panicles based on size, density, and deformability using dean flow fractionation, inertial separation, and other phenomena. Size-based separation can be performed using a separator, such as a filter membrane type setup, or a microfluidic channel containing obstacles that separate particles based on size (see, e.g. FIGS. 4 and 5). Size-based separation may also be performed in the absence of bound beads, and based on the actual difference in cell size alone.

3. Optionally, immunomagnetic separation may be performed on the same starting cell population. Immunomagnetic separation may be performed either before or after size-based separation. The same beads used for size-enhancement may be used for magnetic separation, or a different population of magnetic beads may be used (see, e.g. FIGS. 4, 5, 6).

4. Separated cells are then analyzed using one or more analysis modalities including: imaging, via a microscope or other device, measuring fluorescent signals from the separated cells, FISH, performing genetic analysis via PCR, rt-PCR, array-based or bead-based sequencing protocols, RNA or DNA analysis, expression analysis, proteomic analysis. Optionally, additional sample preparation steps are performed before the final analysis steps, which may include: removing negative fraction cells, segregating and analysis of single cells separately, cell lysis, and/or culturing of viable separated cells either on or off-chip.

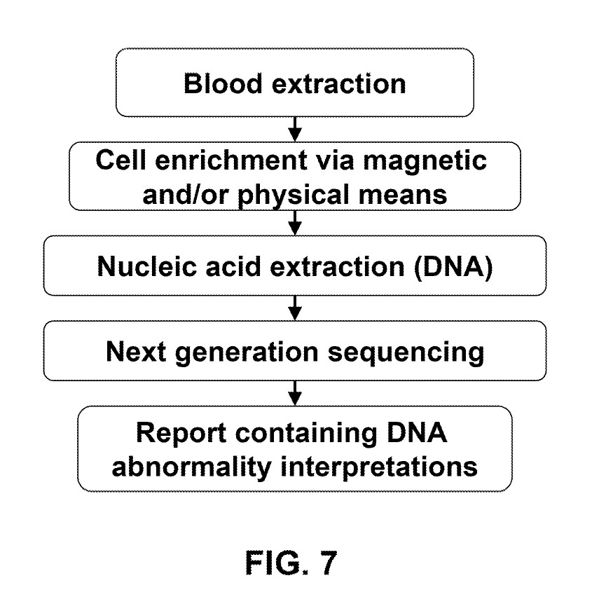

5. Preferred analysis methods include next generation sequencing (NGS), enabled by the higher purity resulting from using 2 or more orthogonal selection criteria (e.g. size and magnetic force). NGS workflows may be augmented by an RNA based assay to determine the presence of CTCs in the isolated sample (e.g. FIGS. 7, 8, and 9).

6. Data resulting from the analysis of said cells can be used diagnostically in a number of ways, including: patient monitoring for minimal residual disease or recurrence, selection of treatment based on known resistance mutations or known sensitizing mutations, a companion diagnostic to newly introduced drug compounds, selection of treatment based on expression profiles that correlate with response to therapy.

Referring to FIGS. 1-9, a method is provided that generally includes coupling magnetic beads to a population of cells in a fluid sample to form magnetically-labeled cells, wherein certain of the magnetically-labeled cells are target cells and others of the magnetically-labeled cells are non-target cells. The fluid sample may generally include any fluid (e.g. gas or liquid). Example liquids include biological fluids such as, but not limited to, blood, urine, sweat, interstitial fluid, or other fluids derived or obtained from a human being or other animal. The fluid sample may include other fluids or additives together with the biological fluid, such as but not limited to, buffer fluids, viscosity-adjusting fluids, or other agents. In some examples the biological fluid may contain multiple cells, some of which are interest for the intended analysis (e.g. target cells) and some of which are not (e.g. background cells). Target cells may include, for example, circulating tumor cells (CTC), circulating fetal cells (CFC), or other cells of interest. Non-target cells may include, for example, white blood cells, or other cells present in the fluid sample which are not of interest to a technique being performed (e.g. sequencing).

In FIGS. 2A, 2B, and 5B-6D, the target cells and the non-target cells may generally be the same size, or in some examples have a significant size overlap in their populations. Magnetic beads may be introduced for binding to cells in the fluid sample. Generally, any magnetic beads may be used, and beads of a variety of sizes may be used, including beads of 1, 2, 3, 4, 5, 6, 7, 8, 9, or 10 μm in size. Magnetic beads may be introduced to the fluid sample and allowed to bind with cells in the sample. In some examples, magnetic beads may be used which are functionalized or otherwise designed for preferential binding to the target cells (e.g. to circulating tumor cells). Although designed for binding to the target cells, some amount of magnetic beads may nonetheless bind to the non-target cells. FIGS. 2A, 2B, and 5B-6D, the target cells 202, 502 602 include many magnetic beads 204, 504, 604 attached to their perimeter, for example, three or more beads attached to their perimeter, whereas the non-target cells 206, 506, 606 generally do not include any magnetic beads attached to their perimeter. A fraction of the magnetic beads 204, 504, 604 may bind non-specifically to the non-target cells 206, 506, 606. As such, in FIGS. 2A, 2B, and 5B-6D, some of the non-target cells 206, 506, 606 include one magnetic bead 204, 504, 604 attached to their periphery to represent the non-specific binding of the magnetic beads to some of the non-target cells. The target cells 202, 502, 602 and the non-target cells 206, 506, 606 including magnetic beads 204, 504, 604 attached to their periphery generally form magnetically-labeled cells that are manipulatable by a magnetic field. The non-target cells 206, 506, 606 not including magnetic heads 204, 504, 604 attached to their periphery generally form non-magnetically-labeled cells and generally are not manipulatable by a magnetic field. Some methods, microfluidic devices, and instruments for magnetic separation of particles from a fluid are described in U.S. Patent Publication No. 2013/0017538 A1, which is hereby incorporated by reference herein in its entirety for all purposes.

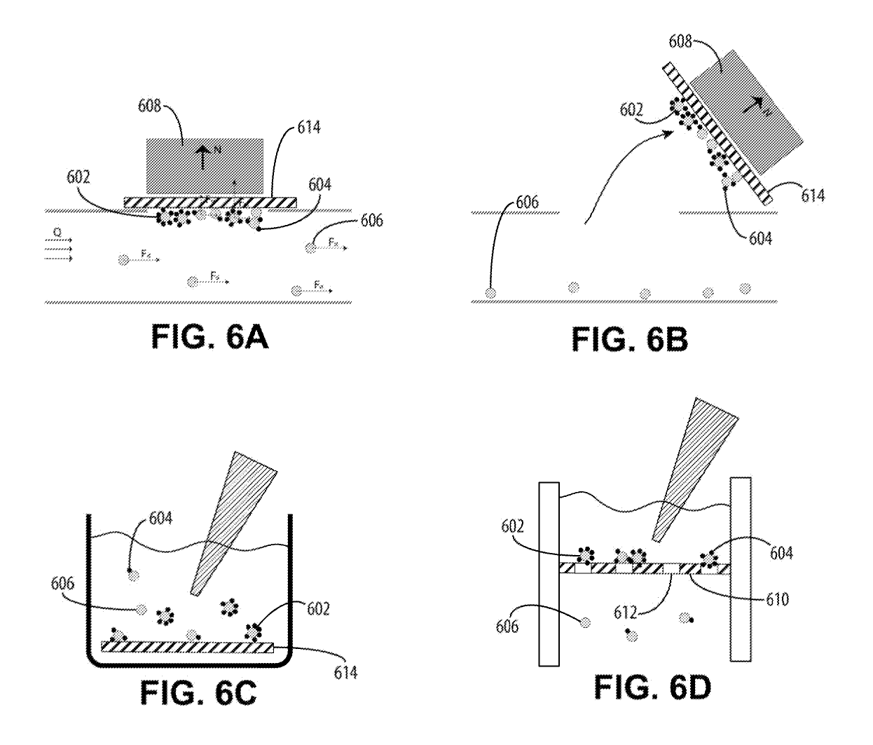

With continued reference to FIGS. 1-9, a method is provided that generally includes magnetically separating the magnetically-labeled cells from the non-magnetically-labeled cells in a fluid sample. Referring specifically to FIGS. 5B and 6A, the magnetically-labeled cells are immobilized on an inner surface of a microfluidic device during flow of the fluid sample through a fluidic pathway of the microfluidic device. In FIGS. 5B and 6A, a magnet 508, 608 is positioned adjacent the fluidic pathway of the microfluidic device and attracts the magnetically-labeled cells to the magnet along an inner surface of the fluidic pathway.

In some embodiments, the non-magnetically-labeled cells are not immobilized by the magnet and flow toward a separator 510 disposed downstream of the immobilized magnetically-labeled cells (see FIGS. 5A-5C). After a suitable period of time, generally sufficient time to allow the non-magnetically labeled cells to exit the magnetic separation region, the magnetically-labeled cells are released from the inner surface of the microfluidic device, for example b removing the magnet 508 or magnetic field, and the magnetically-labeled cells flow toward the separator 510. The magnetically-labeled cells may be immobilized for a period of time such that the non-magnetically-labeled cells are sufficiently downstream of the magnetically-labeled cells to ensure the non-magnetically-labeled cells encounter the separator before the magnetically-labeled cells encounter the separator.

With continued reference to FIGS. 1-9, a method is provided that generally includes separating the target cells from the non-target cells of the magnetically-labeled cells based on a size difference between the magnetically-labeled target cells and the magnetically-labeled non-target cells. Accordingly, a separator 210, 510, 610 may be provided. Any of a variety of separators may be used, including but not limited to a substrate having openings or pores of a threshold size, such that cells greater than the threshold size may not pass through, while those less than the threshold size may pass through. Other separators which may separate cells less than a threshold size from cells greater than a threshold size include spiral fluidic channels. The separator 210, 510, 610 generally includes apertures 212, 512, 612 or other features sized to permit the non-magnetically-labeled cells and the magnetically-labeled non-target cells to pass through the apertures 212, 512, 612 and continue flowing downstream beyond the separator 210, 510, 610 and to prevent the magnetically-labeled target cells from passing through the apertures.

Accordingly, example separators 210, 510, 610 generally capture the magnetically-labeled target cells on an upstream side of the separator 210, 510 610. To remove the magnetically-labeled target cells from the upstream side of the separator 210, 510, 610, the direction of fluid flow Q through the separator may be reversed to flow the magnetically-labeled target cells toward an inlet of the microfluidic device to a location where the cells may be removed from the microfluidic device. The captured magnetically-labeled target cells may be sequenced, as described more fully in other portions of this application.

Referring to FIGS. 6A and 6B, in some embodiments the fluidic pathway includes a removable section 614. As shown in FIGS. 6A and 6B, the removable section 614 may cover an opening formed in the wall of the microfluidic device defining the fluidic pathway. The magnet 608 may be positioned along a top surface of the removable section 614 to attract the magnetically-labeled cells. As schematically represented in FIGS. 6A and 6B, the magnetically-labeled cells may be immobilized on a bottom surface of the removable section 614 of the fluidic pathway of the microfluidic device. The removable section 614 and the immobilized cells may be removed from the microfluidic device (see FIG. 6B) and positioned within a fluid container (see FIG. 6C). As represented in FIGS. 6B and 6C, one or more non-magnetically-labeled cells may be immobilized on the bottom surface of the removable section 614, for example by being trapped between one or more magnetically-labeled cells and the bottom surface of the removable section 614. From the fluid container depicted schematically in FIG. 6C, the cells may be placed on top of a separator 610 (see FIG. 6D). As represented in FIG. 6D, the magnetically-labeled target cells generally are captured on a top surface of the separator 610, whereas the magnetically-labeled non-target cells and the non-magnetically-labeled cells pass through the separator 610. The captured magnetically-labeled target cells may be sequenced, as described more fully in other portions of this application.

With reference to FIGS. 5A-6B, an example microfluidic device 516 is provided. The device 516 generally includes one or more inputs 518, one or more outputs 520, and a fluidic pathway 522 extending between the one or more inputs 518 and the one or more outputs 520. As represented schematically in FIG. 5A, the fluidic pathway 522 generally traverses a magnetic isolation region 524 and a size-based isolation region 526. Referring to FIGS. 5A, 5B, and 6A, the magnetic isolation region 524 generally includes a magnet 508 positioned to separate magnetically-labeled cells from non-magnetically labeled cells in the magnetic isolation region 524. Referring to FIG. 6B, the magnetic isolation region 524 may include removable wall section 614 of the microfluidic device. Referring to FIGS. 5A and 5C, the size-based isolation region 526 may be positioned downstream of the magnetic isolation region 524. Referring to FIG. 5C, the size-based isolation region 526 may include a separator 510. The separator 510 may extend across an entire cross section of the fluidic pathway 522 of the microfluidic device 516 and may define .multiple apertures 512 extending through the separator 510 (see FIG. 5C). The apertures 512 may extend lengthwise parallel to a direction of fluid flow 0 in the fluidic pathway 522 (see FIG. 5C).

With continued reference to FIG. 5C, the separator 510 may be configured to separate cells less than a threshold size from cells greater than a threshold size. The threshold size generally is greater than a size of some of the magnetically-labeled non-target cells but less than a size of some of the magnetically-labeled target cells. In some embodiments, the threshold size is greater than a size of a majority of the magnetically-labeled non-target cells but less than a size of a majority of the magnetically-labeled target cells. With reference to FIGS. 4A-4C, a schematic representation of the relative sizes of example target cells and non-target cells are provided. In FIGS. 4A-4C, the target cells are represented by circulating tumor cells (CFCs), and the non-target cells are represented by white blood cells (WBCs).

FIG. 4A generally represents the relative sizes of the CTCs and the WBCs without magnetic beads bound to the CTCs or WBCs. As shown in FIG. 4A, CTCs have an average size that is generally larger than WBCs. However, there is a substantial size overlap that prevents efficient size-based separation. In FIG. 4A, a filter captured fraction is represented by the rectangular, cross-hatched area, which schematically indicates that an aperture of the separator sized to capture a minimal amount of WBCs to increase a purity level of CFCs relative to the total captured cells would capture less than half of the CTCs. FIG. 4B generally represents the relative sizes of the CTCs and the WBCs after magnetic beads are coupled to the cells. As represented schematically in FIG. 4B, the beads primarily attach to the CTCs, thereby increasing the apparent or effective size of the CTCs relative to the WBCs. By binding beads to the CTCs, the size overlap between the CTCs and the WBCs is reduced, enabling efficient size-based separation (see FIG. 4B). As represented in FIG. 4B, by increasing the effective size of the CTCs with the beads, the same threshold size of the separator captures a majority of the CTCs and only a minimal amount of the WBCs. As represented schematically in FIG. 4C, the concentration of WBCs in the captured cells may be further reduced by performing a magnetic separation step, for example by using same beads bound to CTCs to increase their effective size, to immunomagnetically deplete the WBC population before the size-based separation. This results in a higher purity of CTCs in the captured sample, which may enable sequencing to be performed on the captured sample. In some examples, performing magnetic separation and size-based separation using magnetic beads coupled to the CTCs reduced the number of WBCs from about 10e7 to about 10e2, and the number of CTCs in the sample was about 50 to about 100, resulting in a significant increase in the concentration of CTCs in the captured sample.

### EXAMPLE EMBODIMENTS

### Example 1

### The Use of Beads to Improve Size-Based Separation

In this example the use of bead-assisted size based separation may provide important improvements over size-based separation alone. Let's suppose we were trying to separate two cell populations (FIG. 4) that were different in the expression of a set of markers A (for example EpCAM EGFR, HER2) as well as mean size differences. Since cell sizes differ significantly within the same population, the two fractions will have a significant overlap, meaning that any filtration that selects only cells above the Y0 line will contain only a small fraction of the target cell population (A) if most of background population (B) is to be removed.

The addition of beads of diameter dY to the mixed population will result in preferential binding of the beads to cells expressing marker A, and an increase in the apparent size of cells that are part of the target population (see. FIG. 2). After bead binding, a filtering operation will be much more effective; most of the cells in the target population are above the size separation cutoff (FIG. 4).

The cells thus obtained (e.g., retained by the filter) may be used for a number of genetic DNA-based, RNA-base or protein based testing to aid in patient treatment decisions.

### Example 2

### A Microfluidic Device Integrating Magnetic and Bead-Enhanced Size Separation

A second example exemplifies integration of the magnetic separation and size-based separation in a microfluidic device (see FIG. 5). Let's suppose we were trying to separate two cell populations that were different in the expression of a set of markers A (for example EpCAM, EGFR, HER2) as well as mean size differences. Since cell sizes differ significantly within the same population, the two fractions will have a significant overlap, meaning that any size-based separation will contain both members of the target cell population (A) but also a significant amount of background population (B). Similarly, for magnetic-bead based separation alone, some non-specific binding, will mean that bead pull-out will again contain an enrichment of population (A) along with a significant background from population (B). The addition of beads of diameter dY to the mixed population will result in preferential binding of the beads to cells expressing marker A, and an increase in the apparent size of cells that are part of the target population (see FIG. 4C). The bead-cell mixing and binding may occur within a first region of the microfluidic device.

This may be followed by size-based separation, whereby a large number of cells in population B are allowed to flow through in the microfluidic device (e.g. either steps or posts or branched/curved channels) while a vast majority of cells A are retained, along with the bound beads. Separation may be obtained via either size exclusion (see FIG. 5) or flow driven inertial separation or dean flow fractionation. The bound beads will have served to increase the apparent size of cells A. Cells can then he released from the size-based separation structures via reversed flow or a pressure increase that will apply larger drag forces and/or increase the size of capture structures in a flexible substrate. The cells (still bound by magnetic beads) will then flow through a second separation region in proximity to a magnet, whereby a magnetic force is applied to the beads. Cells of population (A) are retained in this region, while cells of population B, that have no beads bound by met the size-based separation requirements, are allowed to flow through. After target cells (A) are immobilized and other cells washed through, the target cells may be allowed to flow again by reducing the magnetic field. Alternately, the cells of interest may be lysed and the lysate collected downstream. The above operations may be reversed, with the separation using magnetic fields preceding the size-based sorting operation.

The cells thus obtained may be used for a number of tests based on either DNA, RNA or protein (or a combination thereof) to aid in patient treatment decisions.

### Example 3

### Using a Removable Channel Wall section and a Filter device to Enhance Separation Capture Efficiency, Purity, and Cell Recovery (See FIG. 6)

This example provides for a modification of the mechanism of achieving both magnetic bead-based and size-based separation of cell populations. Let's suppose again that the sample presents a mix of the target cell population A and the background cell population B. Cells are pre-labeled with beads that provide a size enhancement and/or magnetic moment in the presence of a field, and preferentially bind cells belonging to population A. Here, cells are introduced into a microfluidic device incorporating a separation chamber.

The positive fraction is immobilized on a substrate in the separation chamber; so only one outlet may be required to receive the negative cell fraction and wash buffer. A magnetic field, source is placed in the vicinity of the separation chamber (see e.g. FIG. 2), where the chamber may simply be a portion of the microfluidic channel. The separation chamber includes a removable substrate that forms at least a part of a wall of the chamber, but may form multiple walls of the separation chamber. As the sample flows through the separation chamber, the positive fraction cells, which are labeled w/magnetic beads, are pulled out of the flow stream and immobilized preferentially onto the separation substrate. After a wash step, the substrate is decoupled from the separation chamber and, along with any bound cells, is placed into a receptacle including a filter substrate (a membrane with pores of controlled size). The sample is then passed through a size-based separation device, either, for example, a filter substrate or a curved flow channel, selectively isolating cells that have a size (as determined by inherent cell size and any bound bead size) larger than a set size cutoff. Cells may be separated based on size of the cell-bead complex, or cell size alone, in the absence of beads.

The cells thus obtained may be used for a number of genetic DNA-based, RNA-base or protein based testing to aid in patient treatment decisions.

### Example 4

### Utilizing Genetic Abnormality and Expression Data From Circulating Cells for Cancer Treatment Decisions

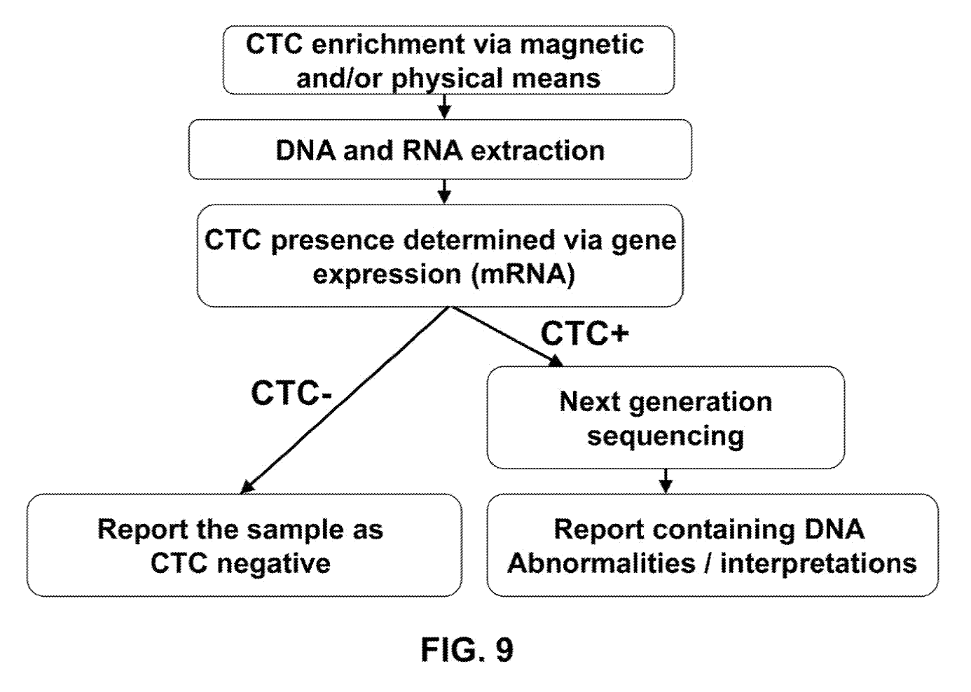

In this example, the isolated rare cell sample is used in order to better determine the course of patient cancer treatment. DNA and RNA may be isolated from the resulting purified cell sample in order to determine the likelihood that the patient will respond to a particular type of therapy, the advantages conferred by a particular therapy type, or the presence of tumor material in the blood stream, and for the risk that said patient's disease will progress. The cells may be purified (e.g. collected using the size and/or magnetic-based methods described herein). DNA and/or RNA from the collected cells may be analyzed to develop or change a course of treatment. For example DNA may be analyzed through next generation sequencing (NGS) methods in order to determine the presence of somatic mutations or alterations such as copy number variations or rearrangements. The methods presented using magnetic- and size-based separation may enable higher purity for circulating tumor cells, which is needed for NGS. The presence of known somatic mutations may be used to determine efficacy of directed therapies, either alone or in conjunction with other biomarkers, such as tumor biopsy data. (See FIGS. 7 and 9). In one example, the presence of KRAS or BRAF mutations may indicate resistance to anti-EGFR agents. The employment of next generation sequencing technologies is specific to the combined affinity/size based separation modality described, because a purity of at least 5% tumor material is required in order to effectively determine the presence of genetic abnormalities. In another example, presence of somatic mutations may be used as a basis for inclusion in ongoing clinical trials. In another example, the presence of somatic mutations may be used to prescribe compounds that were originally developed for a different tissue of origin, in a pathway-dependent approach. Despite the improved purity of tumor and other rare cell samples resulting from multiparametric enrichment, it may be beneficial to look at the resulting samples using low error sequencing methods, such as single molecule barcoding (labeling each DNA molecule with a unique barcode prior to amplification and sequencing steps) and single cell sequencing, as a final readout of the enriched samples.

In cases where no somatic mutations are detected, expression data for tumor cell markers may be used to determine if tumor cells were isolated from the blood sample and tumor-derived present in the final sample; one or more of the following RNA based markers may be used for this purpose: cytokeratin, Ep-CAM, HER2, EGFR, Survivin, hTERT, CK-7, TTF-1, TSA-9, Pre-proGRP, HSFIB1, UCHL1, MUC-1. The presence of tumor cells may be determined by calculating a ‘tumor score’ that includes multiplying the expression level of one or more markers with an individual coefficient and comparison of the tumor score with a pre-determined threshold. For example, a tumor score may be calculated according to the following formula: Score=(expression gene 1)*coefficient 1+(expression gene 2)*coeff. 2+ . . . +(expression gene n) & coefficient n. If the Score is greater than the tumor threshold, the presence of CTCs is confirmed. (See, e.g. FIG. 9)

### Example 5

### Using Gene Expression Data From Circulating Cells for Cancer Treatment Decisions

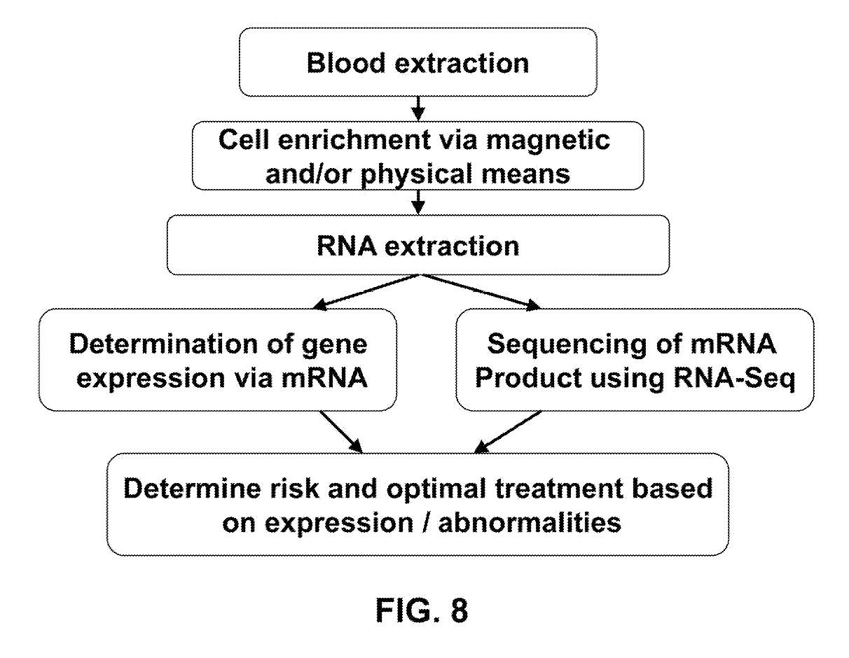

In this example the abundance of mRNA copies of a number of specified genes (gene panel) is used to make a treatment decision (see FIG. 8). For example, for breast cancer, a combination of the following markers may be used: CK19, CK20, CK8, SCGB2A2, MUC1, EpCAM, BIRC5, ERBB2, MRP1,2,4,5,7; dCK, ALDH1, MBG1, MAGEA3, hMAM, CCNE2, DKFZp762E1312, EMP2, MAL2, PPIC, SLC6A, B305D-C, B726P, GABA AP, SCGB2, TFF1, TFF3.

For lung cancer, a combination of the following markers may be used: BIRC5, hTERT, TTF-1, FN1, PGP9.5, TSA-9 (FAM83A), Pre-proGRP, hMTH1(NUDT1), SP-D, ITGA11, COL11A1, LCK, RND3, WNT3a, ERBB3, BAG1, BRCA1, CDC6, CDK2AP1, FUT3, IL11, SH3BGR, EGFR, c-Met, MAGE-A3, CK-19, CK-20, CK-7, EpCAM, CD45

For prostate cancer, a combination of the following markers may be used: CK-19, CK-20, CK-7, EpCAM, CD45, EGFR, PSMA, PSA, AR, HPN, HK2, PSGR, MGB1, MGB2, AZGP1, KLK2, SRD5A2, FAM13C, FLNC, GSN TPM2, GSTM2, TPX2.

The patient risk of progression and/or prognosis may be determined by calculating a ‘tumor score’ that includes multiplying the expression level of one or more markers with an individual coefficient and comparison of the tumor score with a pre-determined threshold. For example, a tumor score may be calculated according to the following formula: Score=(expression gene 1)*coefficient 1+(expression gene 2)*coeff. 2+ . . . +(expression gene n)*coefficient n. Risk is then assessed based on the overall score.

Further, the patient's benefit from a particular systemic treatment (e.g. chemotherapy) or localized treatment (e.g. surgery) may be assessed by calculating a ‘tumor score’ that includes multiplying the expression level of one or more markers with an individual coefficient and comparison of the tumor score with a pre-determined threshold. For example, a tumor score may be calculated according to the following formula: Score=(expression gene 1)*coefficient 1+(expression gene 2)*coefficient 2+ . . . (expression gene n)* coefficient n. Depending on the results, the patient may be assigned a particular adjuvant therapy, or the timeline of localized treatment may be determined.

Rare cell expression profiles may be used as a stand alone test, or in conjunction with tissue based test results or other biomarkers, such as PSA score.

The above examples detail some of the preferred embodiments of the invention. A number of combinations or variations of the above can be envisioned as well, depending on the application requirements. The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, it should be understood that various features of the certain aspects, embodiments, or configurations of the disclosure may he combined in alternate aspects, embodiments, or configurations. Moreover, the following claims are hereby incorporated into this Detailed Description by this reference, with each claim standing on its own as a separate embodiment of the present disclosure.

1. A method comprising:
coupling beads to a population of target cells based on antibody binding in a fluid sample to form target cell-bead aggregates having a larger size than a population of non-target cells in the fluid sample; and
separating the target cell-bead aggregates from the non-target cells based on a size difference between the target cell-bead aggregates and the non-target cells.

2. The method of claim 1, further comprising magnetically separating the target cell-bead aggregates from the non-target cells in the fluid sample.

3. The method of claim 2, further comprising sequencing DNA or mRNA from the target cell-bead aggregates.

4. A method comprising:
coupling magnetic beads to a population of cells in a fluid sample to form magnetically-labeled cells, wherein certain of the magnetically-labeled cells are target cells and others of the magnetically-labeled cells are non-target cells;
magnetically separating the magnetically-labeled cells from non-magnetically-labeled cells in the fluid sample; and
separating the target cells from the non-target cells of the magnetically-labeled cells based on a size difference between the magnetically-labeled target cell-bead aggregates and the magnetically-labeled non-target cells.

5. The method of claim 4, further comprising sequencing DNA or mRNA from the magnetically-labeled target cells.

6. The method of claim 5, wherein the magnetic separation step comprises immobilizing the magnetically-labeled cells on an inner surface of a microfluidic device during flow of the fluid sample through a fluidic pathway of the microfluidic device.

7. The method of claim 6, further comprising flowing a portion of the fluid sample through a separator disposed downstream of the immobilized magnetically-labeled cells.

8. The method of claim 7, further comprising:
releasing the magnetically-labeled cells from the inner surface of the microfluidic device; and
flowing the magnetically-labeled cells toward the separator.

9. The method of claim 7, wherein the separating the target cells from the non-target cells step comprises capturing the magnetically-labeled target cells on an upstream surface of the separator.

10. The method of 6, further comprising sequencing DNA or mRNA from the magnetically-labeled target cells.

11. The method of claim 6, wherein the immobilizing step comprises immobilizing the magnetically-labeled cells on a bottom surface of a removable section of the fluidic pathway of the microfluidic device.

12. The method of claim 11, further comprising:
removing the removable section with the immobilized magnetically-labeled cells from the microfluidic device; and
placing the magnetically-labeled cells on top of a separator.

13. The method of claim 12, wherein the separating the target cells from the non-target cells step comprises capturing the magnetically-labeled target cells on a top surface of the separator.

14. The method of claim 11, further comprising sequencing DNA or mRNA from the magnetically-labeled target cells.

15. The method of claim 4, wherein the fluid sample is a blood sample, and further comprising separating a buffy coat from the blood sample prior to coupling magnetic beads to the population of cells.

16. The method of claim 1, wherein the target cells are tumor cells.

17. A microfluidic device comprising:
an input;
an output; and
a fluidic pathway extending between the input and the output, the fluidic pathway traversing a magnetic isolation region and a size-based isolation region, wherein the magnetic isolation region includes a magnet positioned to separate magnetically-labeled cells from non-magnetically labeled cells in the magnetic isolation region, and wherein the size-based isolation region is downstream of the magnetic isolation region and includes a separator configured to separate cells less than a threshold size from cells greater than a threshold size, wherein the threshold size is greater than a size of some magnetically-labeled non-target cells but less than a size of some magnetically-labeled target cells.

18. The microfluidic device of claim 17, wherein the threshold size is greater than a size of a majority of the magnetically-labeled non-target cells but less than a size of a majority of the magnetically-labeled target cells.

19. The microfluidic device of claim 17, wherein the magnetic isolation region includes a removable wall section of the microfluidic device.

20. The microfluidic device of claim 17, wherein the separator extends across an entire cross section of the fluidic pathway.

21. The microfluidic device of claim 17, wherein the separator defines multiple apertures extending lengthwise parallel to a direction of fluid flow in the fluidic pathway.
